(19) United States
(12) Patent Application Publication
(10) Pub. No.: US 2020/0254104 A1
(43) Pub. Date: Aug. 13, 2020

(54) METHODS OF MAKING AND USING PHYTOCANNABINOIDS COMPLEXED WITH A PROTEIN, PEPTIDE, AMINO ACID, POLYSACCHARIDE, DISACCHRIDE, OR MONOSACCHARIDE

(71) Applicant: John Chancey, Edmond, OK (US)

(72) Inventor: John Chancey, Edmond, OK (US)

(21) Appl. No.: 16/787,460

(22) Filed: Feb. 11, 2020

Related U.S. Application Data
(60) Provisional application No. 62/803,694, filed on Feb. 11, 2019.

(51) Int. Cl. A61K 47/54; A61K 47/64; A61K 36/185; A61K 31/12

(52) U.S. Cl. CPC A61K 47/549; A61K 31/12; A61K 36/185; A61K 47/64

(57) ABSTRACT

The present invention provides a cannabinoid composition comprising a cannabinoid bound to a peptide through a linker to form a cannabinoid-peptide complex, wherein the peptide comprises at least 1 amino acids.

### CROSS-REFERENCE TO RELATED APPLICATIONS

This application claims priority based on U.S. Provisional Application No. 62/803,694, filed Feb. 11, 2019. The contents of which is incorporated by reference in its entirety.

### TECHNICAL FIELD OF THE INVENTION

The present invention is in the field of formulations of Cannabis spp. derived Phytocannabinoids (cannabinoids), and more specifically, to a cannabinoid (CBND) complexed with a protein, peptide, amino acid, polysaccharide, disaccharide, or monosaccharide used in nutraceuticals.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with cannabinoids complexed with a protein, peptide, amino acid, polysaccharide, disaccharide, or monosaccharide used in nutraceuticals. Over 100 cannabinoids have been identified from Cannabis sativa. The cannabinoids include, but not limited to, CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran), THC (tetrahydrocannabinol). The industrial hemp cultivar of the Cannabis sativa contains higher concentrations of the CBND cannabidiol and very low concentrations of tetrahydrocannabinol (THC). THC has a much higher concentration and is the psychoactive component of another cultivar (marijuana) of Cannabis sativa. Industrial hemp extract averages less than 0.3% THC and up to 40% non-THC phytocannabinoids. Cannabidiol (CBD) is the most common non-THC cannabinoid with a concentration of 10-20% of the total CBND content. CBD is extracted from the stalk and seeds of the male Cannabis sativa. THC is extracted primarily from the flowers (trichomes) of the female Cannabis sativa plant. Today there is a growing public awareness of health benefits derived from Cannabis spp., and cannabinoids, including Cannabidiol (CBD). Cannabinoids have been associated with relieving inflammation, anxiety, pain and used as an anti-oxidant. However, cannabinoids (including cannabidiol and tetrahydrocannabinol) are insoluble in water and have poor bioavailability when taken orally. Improvement in solubility and bioavailability may be achieved with complexing CBNDs with proteins, peptides, amino acids, polysaccharides, disaccharides, or monosaccharides used in nutraceuticals.

### BRIEF SUMMARY OF THE INVENTION

In the present invention a cannabinoid (CBND) complexes with proteins, peptides, amino acids, polysaccharides, disaccharides, monosaccharides, amino sugars, glycosaminoglycans, glycol-proteins. Also disclosed are methods of preparing a CBND complex, comprising obtaining a CBND (hemp extract, Cannabis sativa extract, a phytocannabinoid, cannabinoid, terpenophenol); obtaining a protein; and mixing the CBND and the protein in a solvent. Also disclosed are methods of treating a subject, the method comprising identifying a subject in need of treatment of a CBND-related disorder and administering to the subject a nutraceutical composition comprising a CBND-protein complex as described. Also disclosed are methods of preparing a CBND complex, comprising obtaining a CBND; obtaining a peptide; and mixing the CBND and the peptide in a solvent. Also disclosed are methods of treating a subject, the method comprising identifying a subject in need of treatment of a CBND-related disorder, and administering to the subject a nutraceutical composition comprising a CBND-peptide complex as described. Also disclosed are methods of preparing a CBND complex, comprising obtaining a CBND; obtaining an amino acid; and mixing the CBND and the amino acid in a solvent. Also disclosed are methods of treating a subject, the method comprising identifying a subject in need of treatment of a CBND-related disorder and administering to the subject a nutraceutical composition comprising a CBND-amino acid complex as described. Also disclosed are methods of preparing a CBND complex, comprising obtaining a CBND; obtaining a polysaccharide; and mixing the CBND and the polysaccharide in a solvent. Also disclosed are methods of treating a subject, the method comprising identifying a subject in need of treatment of a CBND-related disorder, and administering to the subject a nutraceutical composition comprising a CBND-polysaccharide complex as described. Also disclosed are methods of preparing a CBND complex, comprising obtaining a CBND; obtaining a disaccharide; and mixing the CBND and the disaccharide in a solvent. Also disclosed are methods of treating a subject, the method comprising identifying a subject in need of treatment of a CBND-related disorder, and administering to the subject a nutraceutical composition comprising a CBND-disaccharide complex as described. Also disclosed are methods of preparing a CBND complex, comprising obtaining a CBND; obtaining a monosaccharide; and mixing the CBND and the monosaccharide in a solvent. Also disclosed are methods of treating a subject, the method comprising identifying a subject in need of treatment of a CBND-related disorder, and administering to the subject a nutraceutical composition comprising a CBND-monosaccharide complex as described. Also disclosed are nutraceutical compositions comprising a CBND complexed with a protein, peptide, amino acid, polysaccharide, disaccharide, or monosaccharide as described and a pharmaceutically acceptable excipient, diluent, or carrier.

The present invention provided a CBND complex comprising a therapeutically effective amount of one or more CBNDs selected from Cannabis spp. derived Phytocannabinoids (cannabinoids) and Terpenophenol; and one or more complexing agents conjugated to a therapeutically effective amount of one or more CBNDs, wherein the one or more complexing agents are selected from proteins, peptides, amino acids, polysaccharides, disaccharides, monosaccharides, amino sugars, glycosaminoglycans, and glycol-proteins, disposed in a pharmaceutically acceptable excipient, diluent, or carrier.

The therapeutically effective amount of one or more CBNDs may be non-covalently conjugated to the complexing agent. The therapeutically effective amount of one or more CBNDs may be 2, 3, 4, 5, 6, or more CBNDs. The proteins may be selected from whey protein isolate, egg protein isolate, oat protein isolate, hemp protein, and Brown rice protein isolate. The one or more complexing agents may be N-acetylglucosamine, glucosamine sulfate or N-acetylgalactosamine, glucuronic acid, iduronic acid, galactose chondroitin, chondroitin sulfate, and glucosamine, glycosaminoglycan. The one or more complexing agents may be cysteine, n-acetyl cysteine, methionine, dl methionine, 1 methionine, tyrosine, taurine. The one or more complexing agents may be glutathione.

The present invention provides a nutraceutical composition comprising a therapeutically effective amount of one or more CBNDs selected from a Cannabis spp. derived phytocannabinoids (cannabinoids) and terpenophenols; and one or more complexing agents conjugated to a therapeutically effective amount of one or more CBNDs, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier.

The present invention provides a method of treating a subject suffering from a CBND-related disorder comprising the steps of: identifying a subject in need of treatment of a CBND-related disorder; and administering to the subject a nutraceutical composition comprising a CBND-acid complex comprising a therapeutically effective amount of one or more CBNDs selected from a Cannabis spp. derived phytocannabinoids (cannabinoids) and terpenophenols; and one or more complexing agents conjugated to a therapeutically effective amount of one or more CBNDs, wherein the one or more complexing agents are selected from proteins, peptides, amino acids, polysaccharides, disaccharides, monosaccharides, amino sugars, glycosaminoglycans, glycol-proteins disposed in a pharmaceutically acceptable excipient, diluent, or carrier.

### BRIEF DESCRIPTION OF THE SEVERAL VIEWS OF THE DRAWINGS

For a more complete understanding of the features and advantages of the present invention, reference is now made to the detailed description of the invention along with the accompanying figures and in which:

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, several terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

As used herein the term “Conjugation” follows any conjugation methodology known to the skilled artisan but generally includes the CBND being solubilized with a solvent (ethanol, methanol, etc.) under heat ˜50° C., pressure, proper pH (depending on CBND) and protected from light while mixing/solubilizing and the mixture is cooled to warmed temperatures (3745° C.). The conjugate material (proteins, polysaccharides, etc.) is added and allowed to mix for a period of time. A vacuum is created to lower boiling point and vaporizing the solvent for removal and drying of the material. In some instances, it is possible to combine two CBNDs with a conjugate material, e.g., Cannabidiol (CBD) and cannabidiolic acid (CBDA) can be mixed and conjugated with polysaccharide, or glucosamine sulfate or chondroitin sulfate or a peptide or an amino acid or a protein; or in another embodiment, cannabigerol (CBG) and cannabinol (CBN) can be mixed and conjugated with polysaccharide, or glucosamine sulfate or chondroitin sulfate or a peptide or an amino acid or a protein; or in another embodiment, Cannabidiol (CBD) and tetrahydrocannabinol (THC) can be mixed and conjugated with polysaccharide, or glucosamine sulfate or chondroitin sulfate or a peptide or an amino acid or a protein; or in another embodiment, curcumin and quercetin can be mixed and conjugated with polysaccharide, or glucosamine sulfate or chondroitin sulfate or a peptide or an amino acid or a protein. In some instances, it is possible to combine more than two CBNDs a with conjugate material, e.g., cannabidiol (CBD), cannabigerol CBG and cannabinol (CBN) can be mixed and conjugated with a polysaccharide, or glucosamine sulfate or chondroitin sulfate or a peptide or an amino acid or a protein; or in another embodiment, Cannabidiol (CBD), cannabigerol CBG and tetrahydrocannabinol (THC) can be mixed and conjugated with a polysaccharide, or glucosamine sulfate or chondroitin sulfate or a peptide or an amino acid or a protein.

In yet another embodiment, it is possible to combine a cannabinoid (CBND) and a polyphenol (derived or selected from a turmeric extract, a curcumin, a curcuminoid, a grapeseed extract, a resveratrol, a milk thistle extract, a green tea extract, a epigallocatechin gallate or a quercetin) and can be mixed and conjugated with polysaccharide, or glucosamine sulfate or chondroitin sulfate or a peptide or an amino acid or a protein. It is possible to combine more than one cannabinoid and more than one polyphenol (derived or selected from a turmeric extract, a curcumin, a curcuminoid, a grapeseed extract, a resveratrol, a milk thistle extract, a green tea extract, a epigallocatechin gallate or a quercetin) and can be mixed and conjugated with polysaccharide, or glucosamine sulfate or chondroitin sulfate or a peptide or an amino acid or a protein.

As used herein the term “CBND” denotes a structural class of mainly natural, but also synthetic or semisynthetic, organic chemicals characterized by the presence of terpenophenolic structures. The number and characteristics of these terpenophenolic structures underlie the unique physical, chemical, and biological properties (e.g., metabolic, toxic, therapeutic, etc.). Examples include (but not limited to), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran) and THC (tetrahydrocannabinol). The general physical properties include water-insoluble, moderately water-insoluble and moderately water-soluble compounds with molecular weight of 500-4000 Da, >12 terpenophenolic hydroxyl groups, and 1-2 aromatic rings per 1000 Da (these are general ranges and may be ±20% and be within the definition of CBND.

As used herein the term “proteins” denotes large biomolecules, or macromolecules, consisting of one or more long chains of amino acid residues and includes natural and synthetic and modified R groups to achieve natural, synthetic or modified amino acids. Proteins include, but not limited to, whey protein isolate, egg protein isolate, oat protein isolate, hemp protein, sunflower protein isolate and brown rice protein isolate. Amino acids include cysteine & n-acetyl cysteine, methionine, dl-methionine, 1-methionine, tyrosine, taurine and the like. N-Acetyl-L-cysteine is the N-acetyl derivative of cysteine. It is used as a mucolytic agent to reduce the viscosity of mucous secretions. It has also been shown to have antiviral effects in patients with HIV due to inhibition of viral stimulation by reactive oxygen intermediates. Methionine is one of nine essential amino acids in humans (provided by food), Methionine is required for growth and tissue repair. A sulphur-containing amino acid, methionine improves the tone and pliability of skin, hair, and strengthens nails. Involved in many detoxifying processes, sulphur provided by methionine protects cells from pollutants, slows cell aging, and is essential for absorption and bio-availability of selenium and zinc. Methionine chelates heavy metals, such as lead and mercury, aiding their excretion. It also acts as a lipotropic agent and prevents excess fat buildup in the liver.

As used herein the term “peptides” denotes small biomolecules, or macromolecules, consisting of one or more short chains of amino acid residues. The term “peptide” in the context of a “peptide compound” or a “peptide complex” is meant as a compound having at least two amino acids linked together by a peptide bond. In some embodiments, the peptide is an oligopeptide, for example a bipeptide, having two amino acids, a tripeptide, having three amino acids, a 4-mer, 5-mer, and the like. In some embodiments, the peptide is an oligopeptide comprises between 2-20 amino acids. In other embodiments, Glutathione is a tripeptide comprised of three amino acids (cysteine, glutamic acid, and glycine) present in most mammalian tissue. Glutathione acts as an antioxidant, a free radical scavenger and a detoxifying agent. Glutathione is also important as a cofactor for the enzyme glutathione peroxidase, in the uptake of amino acids, and in the synthesis of leukotrienes. As a substrate for glutathione S-transferase, this agent reacts with several harmful chemical species, such as halides, epoxides and free radicals, to form harmless inactive products. In erythrocytes, these reactions prevent oxidative damage through the reduction of methemoglobin and peroxides. Glutathione is also involved in the formation and maintenance of disulfide bonds in proteins and in the transport of amino acids across cell membranes.

As used herein the term “carrier” denotes a chemical compound that facilitates the incorporation of a compound into cells or tissues. For example, dimethyl sulfoxide (DMSO) is a commonly utilized carrier as it facilitates the uptake of many organic compounds into the cells or tissues of an organism. A common carrier is water, where an aqueous solution of the product of interest is prepared and administered to a subject.

As used herein the term “diluent” denotes chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

As used herein the term “physiologically acceptable” denotes a carrier or diluent that does not abrogate the biological activity and properties of the compound.

As used herein the term “subject” denotes an animal, preferably a mammal, and most preferably a human, who is the object of treatment, observation or experiment. The mammal may be selected from the group consisting of mice, rats, rabbits, guinea pigs, dogs, cats, sheep, goats, cows, pigs, horses, primates, such as monkeys, chimpanzees, and apes, and humans. Other animals include wildlife (deer, elk, moose, bear, lion, rhinoceros, elephant, etc.), avian (birds, poultry, chicken, turkey, duck, etc.), reptiles (snake, turtle, tortoise, lizard, etc.) and fish (freshwater, saltwater, etc.).

As used herein the term “therapeutically effective amount” denotes an amount of the CBND complexed with a protein, peptide, amino acid, polysaccharide, disaccharide or monosaccharide that elicits the biological or medicinal response indicated. This response may occur in a tissue, system, animal or human that is being sought by a researcher, veterinarian, medical doctor or other clinician, and includes alleviation of the symptoms of the disease being treated.

As used herein the term “treat,” “treating,” “treatment,” or any other variation thereof, does not indicate the complete cure from a disorder. Any amelioration of alleviation of the symptoms of a diseases or disorder to any degree, or any increase in the comfort of the subject, is considered treatment.

As used herein the term “Glycosaminoglycans” denotes (GAGs) or mucopolysaccharides are long unbranched polysaccharides consisting of a repeating disaccharide unit. The repeating unit consists of an amino sugar (N-acetylglucosamine, glucosamine sulfate or N-acetylgalactosamine) along with an uronic sugar (glucuronic acid or iduronic acid) or galactose. For example, chondroitin and glucosamine. Chondroitin sulfate is a sulfated glycosaminoglycan (GAG) composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Chondroitin sulfate is an important structural component of cartilage and provides much of its resistance to compression. Glucosamine is commonly used as a treatment for osteoarthritis. It is an amino sugar and a prominent precursor in the biochemical synthesis of glycosylated proteins and lipids. Since glucosamine is a precursor for glycosaminoglycans, and glycosaminoglycans are a major component of joint cartilage, supplemental glucosamine may help to rebuild cartilage and treat arthritis. Other polysaccharides include fucoidan (sulfated polysaccharide) obtained from brown algae and brown seaweed.

Thus, in one aspect, disclosed herein are CBND-protein complex, or CBND-peptide complex or CBND-amino acid complex or CBND-polysaccharide complex or CBND-disaccharide or CBND-monosaccharide complex comprising a CBND compound linked to a protein compound or peptide compound or an amino acid or a polysaccharide compound or a disaccharide compound or a monosaccharide. In other embodiments, the peptide compound is a protein or a protein fragment. In some embodiments, a protein is naturally occurring and is a full sequence polypeptide expressed by a cell. In other embodiments, a protein is a synthetic protein having a sequence that is not found in nature. In some embodiments, the synthetic protein is expressed by a cell using recombinant technologies, whereas in other embodiments, the synthetic protein is synthesized using a peptide synthesizer. A protein fragment is an oligo- or polypeptide having a sequence identical to a sequence fragment found in a protein.

In some embodiments, the CBND compound is linked covalently to a protein compound or peptide compound or an amino acid or a polysaccharide compound or a disaccharide compound or a monosaccharide. In these embodiments, the CBND compound is either bound directly to an amino acid of the peptide, or is bound through a linker compound. In some embodiments, the linker is an alkyl, alkenyl, or alkenyl moiety, which may be substituted with a substituent selected from the group consisting of —OH, —SH, —SO, —COOH, —N—C(O)H, —N—C(O)OH, —C(O)NH, and the like. In some embodiments, the linker is bound to the amino acid or the CBND compound through a substituent. In other embodiments, the CBND compound is linked by hydrogen bonding to the peptide compound to form the complex. In yet other embodiments, the CBND compound is linked by electrostatic forces to the protein compound (or peptide compound or an amino acid or a polysaccharide compound or a disaccharide compound or a monosaccharide) to form the complex. In yet other embodiments, the CBND compound is linked by lipophilic interactions (e.g., van der Waals forces) to the protein compound (or peptide compound or an amino acid or a polysaccharide compound or a disaccharide compound or a monosaccharide) to form the complex. In some embodiments, the peptide is a full-length protein. In certain embodiments, the protein is one that is found in the serum of a mammal. In other embodiments, the protein is derived from an animal source other than a mammal. In still other embodiment, the protein is derived from plants, such as grains, legumes, fruits, vegetables, and the like.

Examples of oligo- and polypeptides and full-length proteins used in the complexes described herein include, but are not limited to whey protein, tumor necrosis factor (TNF-α); cyclooxygenase (COX) (including COX-1 and COX-2); al-acid glycoprotein (AGP) (also known as orosomucoid); myeloid differentiation protein 2 (MD-2); any one of the group of enzymes called histone acetyl-transferases (HATs), such as p300/CBP; any one of the group of enzymes called histone deacetylases (HDAC); glyoxalase I (GLOI); xanthine oxidase (XO); a proteasome; sarco (endo) plasmic reticulum Ca²⁺ ATPase (SERCA); human immunodeficiency virus type 1 (HIV-1) protease; any one of the DNA methyltransferases (DNMTs), for example DNMT1; DNA polymerase (pol) λ; any one of the ribonucleases (RNases), for example RNase A; any one of the lipoxygenases (LOXs); any one of the matrix metalloproteinases (MMPs); lysozyme; any one of the protein kinase C (PKC) family of enzymes; cellular sarcoma (c-Src); glycogen synthase kinase (GSK)-3β; ErbB2; phosphorylase kinase; any one of the protein reductases, for example thioredoxin reductase (TrxR) and aldose reductase (ALR2); thioredoxin reductase; any one of the caseins; human serum albumin (HSA); bovine serum albumin (BSA); fibrinogen; β-lactoglobulin (β-LG); α-lactalbumin; human serum immunoglobulin (Ig); FtsZ; transthyretin (TTR); glutathione (GSH); and Kelch-like ECH-associated protein 1 (Keap1).

In some embodiments, the CBND-protein complex is a complex of CBND and whey protein isolate or a brown rice protein isolate. In certain embodiments the CBND is a terpenophenol, phytocannabinoid, Cannabis spp. extract, hemp, hemp extract, cannabidiol, or a tetrahydrocannabinol. In certain embodiments, the whey protein is a milk-derived whey protein, or the brown rice protein is a plant derived protein. Milk whey protein is a mixture of β-lactoglobulin (˜65%), α-lactalbumin (˜25%), bovine serum albumin (˜8%), and immunoglobulins. In some of these embodiments, the complex is formed by mixing the CBND and the whey protein isolate in ethanol. Thus, in these embodiments, there is no covalent linkage between the CBND and the whey protein. In certain embodiments, the ratio of CBND to whey protein or brown rice protein is 1:20 w/w. In other embodiments the ratio of a CBND to whey protein or brown rice protein is 1:≥40 and 1:≤50 w/w or CBND to whey protein or brown rice protein in any increment between 1:>10 and 1:<40. In other embodiments the ratio of CBND to whey protein or brown rice protein is 1:50 w/w. In some embodiments, the whey protein is obtained from a commercially available source, which comprises 85-90% whey protein in the available powder. In some embodiments, the brown rice protein is obtained from a commercially available source, which comprises 80-90% brown rice protein in the available powder. In some embodiments, the CBND is a terpenophenol, phytocannabinoid, Cannabis spp. extract, hemp, hemp extract, cannabidiol, or a tetrahydrocannabinol and is obtained from a commercially available source.

In some embodiments, the CBND-protein complex is a complex of CBND and egg protein isolate or a fish protein isolate. In certain embodiments the CBND is a terpenophenol, phytocannabinoid, Cannabis spp. extract, hemp, hemp extract, cannabidiol, or a tetrahydrocannabinol. In certain embodiments, the protein is an egg protein or fish protein or other animal derived protein. In some of these embodiments, the complex is formed by mixing the CBND and the egg protein isolate in ethanol. Thus, in these embodiments, there is no covalent linkage between the CBND and the egg protein. In certain embodiments, the ratio of CBND to egg protein or fish protein is 1:20 w/w. In other embodiments the ratio of a CBND to egg protein is 1:≥40 and 1:≤50 w/w or CBND to egg protein or fish protein in any increment between 1:>10 and 1:<40. In other embodiments the ratio of CBND to egg protein is 1:50 w/w. In some embodiments, the fish protein is obtained from a commercially available source, which comprises 80-90% fish protein in the available powder. In some embodiments, the CBND is a terpenophenol, phytocannabinoid, Cannabis spp. extract, hemp, hemp extract, cannabidiol, or a tetrahydrocannabinol and is obtained from a commercially available source.

In some embodiments, the CBND-protein complex is a complex of CBND and brown rice protein. In certain embodiments the CBND is a terpenophenol, phytocannabinoid, Cannabis spp. extract, hemp, hemp extract, cannabidiol, or a tetrahydrocannabinol. In certain embodiments, the brown rice isolate protein is a plant derived protein. In some of these embodiments, the complex is formed by mixing the CBND and the brown rice protein in ethanol. Thus, in these embodiments, there is no covalent linkage between the CBND and the brown rice protein (isolate). In certain embodiments, the ratio of CBND to brown rice protein is 1:<100 w/w. In other embodiments the ratio of a CBND to brown rice protein is 1:≥50 and 1:≤100 w/w or CBND to brown rice protein in any increment between 1:>40 and 1:<50. In some embodiments, the brown rice protein is obtained from a commercially available source, which comprises 80-90% brown rice protein isolate in the available powder. In some embodiments, the CBND is a terpenophenol, phytocannabinoid, Cannabis spp. extract, hemp, hemp extract, cannabidiol, or a tetrahydrocannabinol and is obtained from a commercially available source.

In another aspect, disclosed herein is a nutraceutical composition comprising a CBND-peptide complex, as described herein, and a pharmaceutically acceptable carrier, diluent, or excipient. In certain embodiments the CBND is a terpenophenol, phytocannabinoid, Cannabis spp. extract, hemp, hemp extract, cannabidiol, or a tetrahydrocannabinol. In another aspect, disclosed herein is a nutraceutical composition comprising a CBND-peptide complex, as described herein, and a pharmaceutically acceptable carrier, diluent, or excipient. In certain embodiments the CBND is a terpenophenol, phytocannabinoid, Cannabis spp. extract, hemp, hemp extract, cannabidiol, or a tetrahydrocannabinol. In certain embodiments the peptide is a tripeptide. In other embodiments the peptide is glutathione. In some embodiments the glutathione which comprises the three amino acids L-cysteine, L-glutamic acid and glycine is obtained from a commercially available source. The sulfhydryl group of cysteine is primarily responsible for the biological activity of glutathione. As an important antioxidant, glutathione can decrease intra-cellular damage caused by ROS (reactive oxidative species). In some of these embodiments, the complex is formed by mixing the CBND and the glutathione in ethanol. Thus, in these embodiments, there is no covalent linkage between the CBND and the glutathione. In certain embodiments, the ratio of CBND to glutathione is 1:<10 w/w. In other embodiments the ratio of a CBND glutathione is 1:≥10 and 1:≤20 w/w or CBND to glutathione in any increment between 1:≥1 and 1:≤20.

In another aspect, disclosed herein is a nutraceutical composition comprising a CBND-amino acid complex, as described herein, and a pharmaceutically acceptable carrier, diluent, or excipient. In certain embodiments the CBND is a terpenophenol, phytocannabinoid, Cannabis spp. extract, hemp, hemp extract, cannabidiol, or a tetrahydrocannabinol. In certain embodiments the amino acid is cysteine. In other embodiments the cysteine amino acid is N-Acetyl-cysteine and is available from a commercially available source. N-acetyl cysteine is a white crystalline powder with a slight odor and sour taste. N-acetyl-cysteine is soluble in water and ethanol. N-acetyl-cysteine molecular formula is C₅H₉NO₃S and a molecular weight of 163.191 g/mol. The sulfhydryl group of cysteine is primarily responsible for the biological activity of N-acetyl-cysteine. In some embodiments the amino acid is methionine. In other embodiments the methionine is DL-methionine or N-acetyl-DL-methionine or L-methionine or D-methionine and is available from a commercially available source. The molecular formula for DL-methionine is C₄H₁₁NO₂S and the molecular weight is 149.208 g/mol. Methionine is an essential amino acid required for growth and tissue repair. In some of these embodiments, the complex is formed by mixing the CBND and the amino acid, n-acetyl-cysteine or DL-methionine, in ethanol. Thus, in these embodiments, there is no covalent linkage between the CBND and the amino acid n-acetyl-cysteine or DL-methionine. In certain embodiments, the ratio of CBND to N-acetyl-cysteine or DL-methionine is 1:1, 1:2, 1:4 or 1:≤10 w/w. In other embodiments the ratio of a CBND to N-acetyl-cysteine or DL-methionine is 1:≥10 and 1:≤20 w/w or CBND to N-acetyl-cysteine or DL-methionine in any increment between 1:≥1 and 1:≤20.

In other embodiments, disclosed herein include a nutraceutical composition comprising a CBND-disaccharide complex, as described herein, and a pharmaceutically acceptable carrier, diluent, or excipient. In certain embodiments the CBND is a terpenophenol, phytocannabinoid, Cannabis spp. extract, hemp, hemp extract, cannabidiol, or a tetrahydrocannabinol. In certain embodiments the disaccharide is chondroitin. In other embodiments chondroitin is chondroitin sulfate and is an animal or plant derived mucopolysaccharide or glycosaminoglycan and is available from a commercially available source. Chondroitin sulfate is a white powder and soluble in water and ethanol. Chondroitin sulfate molecular formula is C₁₃H₂₁NO₁₅S and a molecular weight of 463.363 g/mol. In some of these embodiments, the complex is formed by mixing the CBND and the chondroitin sulfate in ethanol. Thus, in these embodiments, there is no covalent linkage between the CBND and chondroitin sulfate. In certain embodiments, the ratio of CBND to chondroitin sulfate is 1:1, 1:2, 1:4 or 1:≤10 w/w. In other embodiments the ratio of a CBND to chondroitin sulfate is 1:≥10 and 1:≤20 w/w or the ratio of a CBND to chondroitin sulfate in any increment between 1:≥1 and 1:≤20.

In other embodiments, disclosed herein include a nutraceutical composition comprising a CBND-monosaccharide complex, as described herein, and a pharmaceutically acceptable carrier, diluent, or excipient. In certain embodiments the CBND is a terpenophenol, phytocannabinoid, Cannabis spp. extract, hemp, hemp extract, cannabidiol, or a tetrahydrocannabinol. In certain embodiments the monosaccharide is glucosamine. In other embodiments glucosamine is glucosamine sulfate and is an animal or plant derived monosaccharide and is available from a commercially available source. Glucosamine sulfate is a white powder and soluble in water and ethanol. glucosamine sulfate molecular formula is C₁₃H₂₁NO₁₅S and a molecular weight of 463.363 g/mol. In some of these embodiments, the complex is formed by mixing the CBND and the glucosamine sulfate in ethanol. Thus, in these embodiments, there is no covalent linkage between the CBND and glucosamine sulfate. In certain embodiments, the ratio of CBND to glucosamine sulfate is 1:1, 12, 1:4 or 1:≤10 w/w. In other embodiments the ratio of a CBND to glucosamine sulfate is 1:≥10 and 1:≤20 and 1:≤50 w/w or the ration of a CBND to glucosamine sulfate in any increment between 1:≥1 and 1:≤50.

In another aspect, disclosed herein is a nutraceutical composition comprising a CBND complexed with a protein, peptide, amino acid, polysaccharide, disaccharide or monosaccharide, as described herein, and a pharmaceutically acceptable carrier, diluent, or excipient. The nutraceutical compositions disclosed herein may be manufactured in a manner that is itself known, e.g., by means of conventional mixing, dissolving, granulating, dragee-making, levigating, emulsifying, encapsulating, entrapping or tableting processes. Nutraceutical compositions disclosed herein thus may be formulated in conventional manner using one or more physiologically acceptable carriers comprising excipients and auxiliaries which facilitate processing of the CBND-conjugate complexed with a protein, peptide, amino acid, polysaccharide, disaccharide or monosaccharide into preparations which can be used nutraceutically or as a food ingredient (e.g., drink mixes, chocolate, gummies, granola, soup mixes, etc.). Any of the well-known techniques, carriers, and excipients may be used as suitable and as understood in the art; e.g., in Remington's Pharmaceutical Sciences, above.

For oral administration, the CBND complexed with a protein, peptide, amino acid, polysaccharide, disaccharide or monosaccharide can be formulated readily by combining the polyphenol complexed with a protein, peptide, amino acid, polysaccharide, disaccharide or monosaccharide with pharmaceutically acceptable carriers well known in the art. Such carriers enable the presently disclosed complexes to be formulated as tablets, pills, dragees, capsules, liquids, gels, syrups, slurries, suspensions and the like, for oral ingestion by a subject. Nutraceutical preparations for oral use can be obtained by mixing one or more solid excipient with the disclosed CBND-conjugate complexes, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Nutraceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Nutraceutical compositions suitable for use in the methods disclosed herein include compositions where the CBND-conjugate complex is contained in an amount effective to achieve its intended purpose. More specifically, a therapeutically effective amount means an amount of the CBND-conjugate complex effective to prevent, alleviate or ameliorate symptoms of disease or prolong the survival of the subject being treated.

Typically, the dose range of the CBND complexed with a protein, peptide, amino acid, polysaccharide, disaccharide or monosaccharide administered to the patient is from about 0.05 to 100 mg/kg of the patient's body weight. The dosage may be one or a series of two or more given in the course of one or more days, as is needed by the patient. In some embodiments, the dosage is between 0.1 mg to 50 mg. In other embodiments, the dosage is between 1 mg to 10 mg. Other dose ranges include between 10 to 50 mg, between 20 to 50 mg, between 30 to 50 mg, between 40 to 50 mg, between 20 to 40 mg, between 10 to 20 mg, between 10 to 30 mg, between 20 to 30 mg, and between 30 to 40 mg. The dose may also be at 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. As used above the dosage refers to active agent dosage and cam be in up to 100 mg or 150 mg. In some embodiments, the dosage is 600 mg s.i.d. (once a day). In another embodiment, the CBND complexed with a protein, peptide amino acid, polysaccharide, disaccharide or monosaccharide when given orally, the total dosage is 600-1200 mg per os b.i.d (twice a day) or 600-1200 mg per os t.i.d. (three times a day) or 600-1200 mg per os or 600-1200 mg per os q.i.d. (four times a day)

In another aspect, disclosed herein is a method of treating a disorder, the method comprising identifying a subject in need thereof and administering to the subject a therapeutically effect amount of a CBND complex as disclosed herein.

In another aspect, disclosed herein is a method of treating a disorder, the method comprising identifying a subject in need thereof and administering to the subject a therapeutically effect amount of a CBND complexed with a protein, peptide, amino acid, polysaccharide, disaccharide or monosaccharide as disclosed herein, where subsequent to the administration, the serum Cₘₐₓ of CBND is >1 ng/ml<2,000 ng/mL. In some embodiments, the serum Cmax of CBND is <0.001% of the administered dose of CBND. The definition of the pharmacokinetic parameter Cₘₐₓ is well-known to those of skill in the art. Briefly, Cₘₐₓ is the maximum observed plasma concentration after a dosage administration.

In another aspect, disclosed herein is a method of preparing a polyphenol complexed with a protein, peptide, amino acid, polysaccharide, disaccharide or monosaccharide, as described above, the method comprising obtaining a polyphenol; obtaining a protein; obtaining a peptide; obtaining an amino acid; obtaining a polysaccharide; obtaining a disaccharide; or obtaining a monosaccharide and mixing the polyphenol and the protein or the peptide or the amino acid or the polysaccharide or the disaccharide or the monosaccharide in a solvent. In some embodiments, the solvent is a polar solvent, while in other embodiments, the solvent is an apolar solvent. In some embodiments, the polar solvent is water, whereas in other embodiments, the polar solvent is an alcohol. In some embodiments, the alcohol is ethanol or methanol.

### Example 1

Preparation of CBND-Glucosamine sulfate Complex. A CNBD-glucosamine sulfate complex was prepared for administration to human subjects. The following materials were used: glucosamine sulfate was 100% by weight, CNBD was 99% by weight and 100% ethyl alcohol. Ratio of CNBD:glucosamine sulfate of 1:10 w/w. A 1% w/v tincture (solution) was prepared by mixing 10 gm cannabidiol isolate powder with 100 grams ethanol. The mixture was placed on a magnetic stirring hot plate, with a speed setting at medium, and temperature setting at 50° C. for 30 minutes or until solution turned clear. To the resulting solution was added 100 gm glucosamine powder. The mixture was placed on a rotary evaporator (rotovap) at slow speed (20-30 rpm), having a water bath temperature of 50° C., and low vacuum for 3-4 hours or until the ethanol was evaporated. Alternatively, the mixture was placed in a lyophilizer. The final product was a fine and white colored powder. The powder re-solubilizes in water with stirring. Alternative method—In a vacuum blender the CBND is added to one kilogram. solvent (methanol, acetate, or ethanol) at the rate of 5 grams per kilogram. The mixture is blended at 50° C. for 60 minutes or until mixture is clear. 500 grams of glucosamine sulfate per kilogram solvent is added and continued mixing at 40° C. for thirty minutes. Low vacuum is applied to remove solvent until dry. Ratio of a single CNBD:glucosamine sulfate of 1 gm CNBD:100 gm glucosamine sulfate powder or 1:100 w/w. The above procedure was repeated, except with 50 g CNBD powder and 1000 g glucosamine sulfate powder. A similar product was obtained with a ration of 1:20 w/w of CNBD to glucosamine sulfate powder. The above procedure was repeated, except with 25 g CNBD powder and 1000 g glucosamine sulfate powder. A similar product was obtained with a ration of 1:40 w/w of CNBD to glucosamine sulfate. The above procedure was repeated, except with 50 g cannabidiol (CBD) powder (99%) and 1000 grams glucosamine sulfate powder. A similar product was obtained with a ration of 1:20 w/w of cannabidiol.

Cannabidiol-chondroitin, cannabidiol-glucosamine, cannabidiol-polysaccharide, cannabidiol-protein, cannabidiol-peptide, cannabidiol-amino acid, cannabinol-chondroitin, cannabinol-glucosamine, cannabinol-polysaccharide, cannabinol-protein, cannabinol-peptide, cannabinol-amino acid, tetrahydrocannabinol-peptide, tetrahydrocannabinol-amino acid, tetrahydrocannabinol-chondroitin, tetrahydrocannabinol-glucosamine, tetrahydrocannabinol-polysaccharide, and tetrahydrocannabinol-protein.

The present invention includes a composition having CBND-protein conjugates. In this embodiment the protein may be a natural or synthetic protein and may be of any length, e.g., dipeptide, tripeptides, polypeptides, oligopeptides, etc. The peptide may be conjugated to CBND, where the peptide is an amino acid. The compositions may be made by solvent assisted blending of CBND-protein conjugates, CBND-peptide conjugates and CBND-amino acid conjugates.

The formulations may include the active agent in communication with a polysaccharide, mucopolysaccharide, glycosaminoglycan, disaccharide, monosaccharide or an amino sugar that is synthetic or naturally occurring. For example, the composition may be CBND-glucosamine conjugates, and CBND-chondroitin conjugates.

In any of the embodiments may include common peptides (proteins) to be used including proteins like whey, brown rice, egg, hemp protein, etc.; amino acids—cysteine, methionine; saccharides to be used include glucosamine, polysaccharide & chondroitin.

The formulations may include cannabidiol-chondroitin conjugates, using chondroitin sulfate—mucopolysaccharide (sulfated glycosaminoglycan) which is a white crystalline powder and 247.30 g/mol, chemical formula C₁₄H₁₉O₁₄S, with a melting point about 190-194° C., is water soluble having a pH 5.5-7.5 and may come from marine or animal sources. Chondroitin sulfate is produced from enzymatic digestion of bovine, poultry, porcine and marine animal cartilaginous tissues. The benefits include as a dietary supplement for joint health since chondroitin sulfate is a major component of cartilage. Loss of chondroitin sulfate from the cartilage is a major cause of osteoarthritis. Methodology/Preparation. Cannabidiol solubility in water is very poor. Organic solvents (methanol, acetone, DMSO, etc.) will increase solubility. An example of the cannabidiol-chondroitin sulfate complex is prepared using the following materials: chondroitin sulfate about 99%; cannabidiol isolate powder about 99% by weight; Ethanol about 95% ethyl alcohol. The composition is processed by blending/Processing: 10 grams cannabidiol with 100 grams ethanol is placed in a rotary mixing vessel at room temperature and medium speed (20 rpm) and blended for 2 hours; 90 grams chondroitin sulfate powder is added with continued mixing for another 30 minutes; and a low vacuum is generated in the vessel to remove the ethanol solvent. The resulting powder is a fine, slightly crystalline, off-white color and able to solubilize in water. The ratio of cannabidiol:chondroitin sulfate is 1:10 w/w.

The formulations include cannabidiol-glucosamine conjugate using glucosamine sulfate, which is a white crystalline powder with a 277.2496 g/mol and a chemical formula C₆H₁₅NO₉S and a melting point about 192° C. The source may be from Marine (shellfish, crustacean) origin—primary and fermentation of grains (primarily corn or wheat). It is slightly water soluble. The benefits include as a dietary supplement for joint health. The methodology/preparation of the cannabidiol-glucosamine sulfate complex is prepared using the following materials: glucosamine sulfate about 99%; cannabidiol isolate powder about 99%; ethanol about 95% ethyl alcohol. The processing is done by blending/processing: 10 grams cannabidiol with 100 grams ethanol is placed in a rotary mixing vessel at room temperature and medium speed (20 rpm) and blended for 2 hours. 90 grams of glucosamine sulfate powder is added with continued mixing for another 30 minutes. A low vacuum is generated in the vessel to remove the ethanol solvent. Resulting powder is a fine, slightly crystalline, off-white color and able to solubilize in water. The ratio of cannabidiol:glucosamine sulfate is 1:10 w/w.

In another instance, a CBND and a polyphenol may be complexed with a protein, peptide, amino acid, polysaccharide, disaccharide, or monosaccharide using the same methods described above. In some embodiments, the polyphenol is a curcuminoid and the CNBD is a cannabidiol isolate powder. The polysaccharide is glucosamine sulfate. Example 1—Preparation of curcuminoid/cannabidiol-glucosamine sulfate complex. A curcumin/cannabidiol-glucosamine sulfate complex was prepared for administration to human and animal subjects. The following materials were used: glucosamine sulfate was 99% by weight, curcumin was 95% curcuminoids by weight, cannabidiol isolate powder 99% by weight and 100% ethyl alcohol. Ratio of curcumin:cannabidiol:glucosamine sulfate is 10:1:40 w/w. A 4% w/v tincture (solution) was prepared by mixing 20 gms curcumin powder and 2 grams cannabidiol extract powder with 100 grams ethanol. The mixture was placed on a magnetic stirring hot plate, with a speed setting at medium, and temperature setting at 50° C. for 30 minutes or until solution turned clear. To the resulting solution was added 80 g glucosamine sulfate powder. The mixture was placed on a rotary evaporator at slow speed (20-30 rpm), having a water bath temperature of 50° C., and low vacuum for 3-4 hours or until the ethanol was evaporated. Alternatively, the mixture was placed in a lyophilizer. The final product was a fine and yellow colored powder. The powder re-solubilizes in water with stirring. Alternative method—In a vacuum blender 40 gm curcumin and 4 gm cannabidiol are added for every liter of solvent (ethanol). The mixture is blended at 50° C. for 60 minutes or until mixture is clear. 160 grams of glucosamine sulfate per liter solvent is added and continued mixing at 40° C. for thirty minutes. Vacuum is applied to reactor to remove solvent until dry.

In another embodiment a CBND and a polyphenol may be complexed with a protein, peptide, amino acid, polysaccharide, disaccharide, or monosaccharide using the same methods described above. In some embodiments, the polyphenol is a curcuminoid and the CNBD is a cannabidiol isolate powder. The protein is whey protein isolate. Example 1—Preparation of curcumin/cannabidiol isolate powder-whey protein isolate complex. A curcumin/cannabidiol isolate powder-whey protein isolate complex was prepared for administration to human and animal subjects. The following materials were used: whey protein isolate was 90% protein by weight, curcumin was 95% curcuminoids by weight, cannabidiol isolate powder was 99% by weight and 100% ethyl alcohol. Ratio of curcumin:cannabidiol isolate powder:whey protein isolate is 4:1:100 w/w. A 0.5% w/v tincture (solution) was prepared by mixing 40 g curcumin powder and 10 gram cannabidiol isolate powder with 1,500 grams ethanol. The mixture was placed on a magnetic stirring hot plate, with a speed setting at medium, and temperature setting at 50° C. for 30 minutes or until solution turned clear. To the resulting solution was added 950 g whey protein isolate powder. The mixture was placed on a rotary evaporator at slow speed (20-30 rpm), having a water bath temperature of 50° C., and low vacuum for 3-4 hours or until the ethanol was evaporated. Alternatively, the mixture was placed in a lyophilizer. The final product was a fine and yellow colored powder. The powder re-solubilizes in water with stirring. Alternative method—In a vacuum blender 20 gm curcumin and 5 gm cannabidiol isolate powder are added per liter solvent (ethanol). The mixture is blended at 50° C. for 60 minutes or until mixture is clear. 425 grams of whey protein isolate per liter solvent is added and continued mixing at 40° C. for thirty minutes. Vacuum is applied to reactor to remove solvent until dry.

Clinical Case Studies—Example 1: A 60 year old male was presented with lower back pain and pain from hip, and foot arthritis. The individual suffers from irritable bowel syndrome (IBD). Several different dosages and formulations were given.

| Formulation and Dosage | Effect | Side effects |
|---|---|---|
| 700 mg conjugate once a day orally | Symptom relief from lower back | Transient headache; sleep |
| for one week - | pain, hip and foot arthritis; IBD | disturbance; temporary mild |
| 80 mg. Curcumin | symptom relief.; significant pain | constipation |
| 20 mg. Cannabidiol | relief with normal work activities | |
| 600 mg. glucosamine sulfate | | |
| 700 mg. conjugate twice a day for two | Symptom relief from lower back | Transient headache; sleep |
| weeks - | pain, foot arthritis, and IBD; | disturbance; mild |
| 10 mg. Cannabidiol | some pain relief with normal | constipation |
| 700 mg. glucosamine sulfate | work activities | |
| 600 mg conjugate twice a day for one | Symptom relief from lower back | Transient headache; sleep |
| week - | pain, foot arthritis, and IBD; | disturbance; temporary |
| 10 mg Cannabidiol | significant pain relief with | constipation |
| 500 mg. Whey protein isolate | normal work activities | |

Clinical Case Studies—Example 2: A 66 year old male was presented with lymphoma and multi-focal enlarged lymph nodes. The patient was administered 800 mg (600 mg. glucosamine sulfate; 80 mg. curcumin; 10 mg. Cannabidiol) conjugate once a day for two weeks. He displayed moderate shrinking of affected lymph nodes after two weeks. Further use of material did not have any further effect. Side effects—none documented

Clinical Case Studies—Example 4: A 52 year old male suffers from pain associated with pelvic arthritis from previous injury. The patient was administered 500 mg (490 mg. glucosamine sulfate; 10 mg. cannabidiol) conjugate once a day for two weeks. There was significant pain relief after one week. The individual has been able to resume normal day to day activities. Side effects—none noted.

Clinical Case Studies—Example 5: A 79 year old female was presented with debilitating arthritis in both ankles. She had pain at rest, and was ambulatory primarily in a wheelchair, with minimal ability to stand or walk with a walker. She used various NSAIDs and prescription anti-pain narcotics daily. The patient was administered 500 mg (490 mg. glucosamine sulfate; 10 mg. cannabidiol) conjugate twice a day for a month. She obtained significant symptom relief from arthritis, including decreased pain and increased mobility without the use of a wheelchair or a walker. She discontinued the use of the narcotics. Side effects—none noted

Clinical Animal Case Studies—Example 1: A 7 year old Golden Retriever 90 lb. spayed female was presented with lameness and lethargy associated with right shoulder arthritis and hip dysplasia. The animal was given 350 mg (300 mg. glucosamine sulfate; 45 mg. curcumin; 5 mg. cannabidiol) conjugate in the feed twice a day for two weeks. The dog resumed normal behavior and significant relief from pain associated with the arthritis. Side effects—none noted.

Clinical Animal Case Studies—Example 3: A 3 year old mix breed 25 lb. spayed female displayed exercise induced lumbar vertebral trauma with moderate pain and lethargy. The dog was administered 150 mg. (125 mg. chondroitin sulfate; 22 mg curcumin; 3 mg cannabidiol) conjugate orally once a day for one month. The dog resumed normal activities without any signs of associated back pain and lethargy.

Clinical Animal Case Studies—Example 4: An 8 month old mix breed 60 lb. neutered male displayed moderate pain and stiffness associated with physitis involving the front limbs. The dog was administered 250 mg. (245 mg. glucosamine sulfate; 5 mg cannabidiol) conjugate orally twice a day for one month. The dog demonstrated relief from the pain and stiffness associated with the physitis.

1. A cannabinoid composition comprising
a cannabinoid bound to a peptide to form a cannabinoid-peptide complex, wherein the peptide comprises at least 1 amino acids.

2. The composition of claim 1, wherein the ratio of cannabinoid to peptide is between 1-50:1-50 w/w (mg:g).

3. The composition of claim 2, wherein the w/w (mg:g) ratio of cannabinoid to peptide is about 1:1; 1:<10; 10:>1; 25:1; 1:25, 50:1 or 1:50. 10:1; 20:1; >50:1; <50:1; 100:1; <100:1, 200:1; <200:1

4. The composition of claim 1, wherein the cannabinoid is bound to the peptide through a linker.

5. The composition of claim 4, wherein the linker is an alkyl, alkenyl, or alkynyl moiety.

6. The composition of claim 4, wherein the linker is a substituted alkyl, a substituted alkenyl, or a substituted alkynyl moiety, wherein the substitution is a —OH, —SH, —COOH, —N—C(O)H, —N—C(O)OH, or —C(O)NH.

7. The composition of claim 1, wherein the cannabinoid is selected from the group consisting of CBND, hemp extract, Cannabis sativa extract, a phytocannabinoid, cannabinoid, terpenophenol.

8. The composition of claim 1, wherein the peptide comprises whey protein isolate, egg protein isolate, oat protein isolate, hemp protein, and Brown rice protein isolate.

9. The composition of claim 4, linker is selected from N-acetylglucosamine, glucosamine sulfate or N-acetylgalactosamine, glucuronic acid, iduronic acid, galactose chondroitin, chondroitin sulfate, and glucosamine, glycosaminoglycan glutathione, cysteine, n-acetyl cysteine, methionine, dl methionine, 1 methionine, tyrosine, or taurine.

10. The composition of claim 9, wherein the cannabinoid and linker comprise Cannabidiol-chondroitin, cannabidiol-glucosamine, cannabidiol-polysaccharide, cannabidiol-protein, cannabidiol-peptide, cannabidiol-amino acid, cannabinol-chondroitin, cannabinol-glucosamine, cannabinol-polysaccharide, cannabinol-protein, cannabinol-peptide, cannabinol-amino acid, tetrahydrocannabinol-peptide, tetrahydrocannabinol-amino acid, tetrahydrocannabinol-chondroitin, tetrahydrocannabinol-glucosamine, tetrahydrocannabinol-polysaccharide, or tetrahydrocannabinol-protein.

11. The composition of claim 1, wherein the peptide is selected from the group consisting of a dipeptide, a tripeptide, an oligopeptide, a polypeptide, a protein, and a protein fragment.

12. The composition of claim 1, wherein the cannabinoid-peptide complex are conjugated by a covalent bond, an ionic interaction, a lipophilic (van der Waals) interaction, or a hydrogen bond.

13. The composition of claim 1, wherein the composition comprises Curcuminoid (a polyphenol), Cannabidiol, and glucosamine sulfate.

14. A therapeutic cannabinoid composition comprising
a cannabinoid bound to a linker that binds to a peptide to form a cannabinoid-peptide complex in a pharmaceutically acceptable excipient, diluent, or carrier, wherein the peptide comprises at least 1 amino acids.

15. The therapeutic cannabinoid composition of claim 14, wherein the cannabinoid is selected from the group consisting of CBND, hemp extract, Cannabis sativa extract, a phytocannabinoid, cannabinoid, terpenophenol.

16. The therapeutic cannabinoid composition of claim 14, wherein the cannabinoid-peptide complex comprises Curcumin, Cannabidiol, and glucosamine sulfate.

17. The therapeutic cannabinoid composition of claim 14, wherein the w/w (mg:g) ratio of cannabinoid to peptide is about 1:1; 1:<10; 10:>1; 25:1; 1:25, 50:1 or 1:50. 10:1; 20:1; >50:1; <50:1; 100:1; <100:1, 200:1; <200:1

18. The therapeutic cannabinoid composition of claim 17, wherein the linker is an alkyl, alkenyl, or alkynyl moiety.

19. The therapeutic cannabinoid composition of claim 17, wherein the linker is selected from N-acetylglucosamine, glucosamine sulfate or N-acetylgalactosamine, glucuronic acid, iduronic acid, galactose chondroitin, chondroitin sulfate, glucosamine, glycosaminoglycan, cysteine, n-acetyl cysteine, methionine, dl methionine, 1 methionine, tyrosine, taurine.

20. The therapeutic cannabinoid composition of claim 14, wherein the peptide is selected from the group consisting of an amino sugar, polysaccharides, monosaccharides, a dipeptide, a tripeptide, an oligopeptide, a polypeptide, a protein, a protein fragment, whey protein isolate, egg protein isolate, oat protein isolate, hemp protein, and Brown rice protein isolate.

21. The therapeutic cannabinoid composition of claim 14, wherein the therapeutic cannabinoid composition comprises Cannabidiol-chondroitin, cannabidiol-glucosamine, cannabidiol-polysaccharide, cannabidiol-protein, cannabidiol-peptide, cannabidiol-amino acid, cannabinol-chondroitin, cannabinol-glucosamine, cannabinol-polysaccharide, cannabinol-protein, cannabinol-peptide, cannabinol-amino acid, tetrahydrocannabinol-peptide, tetrahydrocannabinol-amino acid, tetrahydrocannabinol-chondroitin, tetrahydrocannabinol-glucosamine, tetrahydrocannabinol-polysaccharide, or tetrahydrocannabinol-protein.
